SEQUENCE LISTING (10 sequences)

SEQ ID NO: 1 - protein, 164 aa, Unknown
  Endolysin derived from Citrobacter Koseri phage CkP1
MNIFKMLRIDEGYDSKIYKDTEGFWTIGIGHLLTRDPSLEVAKRELDKLVGRKCNGQITQSEAEKIFADDVDKAINGIKKNASLKPVYDSLDGDDPRQAALINMVFQMGVAGVAGFTNSMRMVKEKRWADAAVNLAQSKWYRQTPNRAKRVIETFRTGTWNAYK

SEQ ID NO: 2 - DNA, 492 bp, Unknown
  Endolysin derived from Citrobacter Koseri phage CkP1
atgaacatttttaaaatgcttcgtatcgatgaaggatacgattctaaaatctataaagataccgaagggttttggactatcggtatcggtcaccttttaactcgcgacccttctcttgaagtagccaaaagagaacttgataaactcgttgggcgtaagtgcaacggtcaaatcactcagtcggaagcagaaaagattttcgctgatgatgttgataaagctattaatggaatcaagaaaaacgcttctctgaaaccggtttatgattctcttgatggagatgatccgcgtcaagctgcactcatcaatatggtatttcaaatgggtgtagctggtgtagctggcttcactaattctatgagaatggtaaaagaaaaacgttgggctgatgcagctgtaaatttagctcaatcaaaatggtatcgtcaaactccgaatcgcgctaaacgagtaattgaaacattccgcactggaacatggaacgcatataaa

SEQ ID NO: 3 - protein, 163 aa, Unknown
  Catalytic domain of endolysin derived from Citrobacter Koseri phage CkP1
MNIFKMLRIDEGYDSKIYKDTEGFWTIGIGHLLTRDPSLEVAKRELDKLVGRKCNGQITQSEAEKIFADDVDKAINGIKKNASLKPVYDSLDGDDPRQAALINMVFQMGVAGVAGFTNSMRMVKEKRWADAAVNLAQSKWYRQTPNRAKRVIETFRTGTWNAY

SEQ ID NO: 4 - DNA, 489 bp, Unknown
  Catalytic domain of endolysin derived from Citrobacter Koseri phage CkP1
atgaacatttttaaaatgcttcgtatcgatgaaggatacgattctaaaatctataaagataccgaagggttttggactatcggtatcggtcaccttttaactcgcgacccttctcttgaagtagccaaaagagaacttgataaactcgttgggcgtaagtgcaacggtcaaatcactcagtcggaagcagaaaagattttcgctgatgatgttgataaagctattaatggaatcaagaaaaacgcttctctgaaaccggtttatgattctcttgatggagatgatccgcgtcaagctgcactcatcaatatggtatttcaaatgggtgtagctggtgtagctggcttcactaattctatgagaatggtaaaagaaaaacgttgggctgatgcagctgtaaatttagctcaatcaaaatggtatcgtcaaactccgaatcgcgctaaacgagtaattgaaacattccgcactggaacatggaacgcatat

SEQ ID NO: 5 - protein, 163 aa, Unknown
  Endolysin derived from Citrobacter Koseri phage CkP1 w/o N-terminal methionine
NIFKMLRIDEGYDSKIYKDTEGFWTIGIGHLLTRDPSLEVAKRELDKLVGRKCNGQITQSEAEKIFADDVDKAINGIKKNASLKPVYDSLDGDDPRQAALINMVFQMGVAGVAGFTNSMRMVKEKRWADAAVNLAQSKWYRQTPNRAKRVIETFRTGTWNAYK

SEQ ID NO: 6 - protein, 162 aa, Unknown
  Catalytic domain of endolysin derived from Citrobacter Koseri phage CkP1 w/o N-terminal methionine
NIFKMLRIDEGYDSKIYKDTEGFWTIGIGHLLTRDPSLEVAKRELDKLVGRKCNGQITQSEAEKIFADDVDKAINGIKKNASLKPVYDSLDGDDPRQAALINMVFQMGVAGVAGFTNSMRMVKEKRWADAAVNLAQSKWYRQTPNRAKRVIETFRTGTWNAY

SEQ ID NO: 7 - protein, 184 aa, Artificial sequence
  Recombninant endolysin derived from Citrobacter Koseri phage CkP1 including N-terminal HisTag
MGSSHHHHHHSSGLVPRGSHMNIFKMLRIDEGYDSKIYKDTEGFWTIGIGHLLTRDPSLEVAKRELDKLVGRKCNGQITQSEAEKIFADDVDKAINGIKKNASLKPVYDSLDGDDPRQAALINMVFQMGVAGVAGFTNSMRMVKEKRWADAAVNLAQSKWYRQTPNRAKRVIETFRTGTWNAYK

SEQ ID NO: 8 - DNA, 552 bp, Artificial sequence
  Recombninant endolysin derived from Citrobacter Koseri phage CkP1 including N-terminal HisTag
atgggcagcagccatcatcatcatcatcacagcagcggcctggtgccgcgcggcagccatatgaacatttttaaaatgcttcgtatcgatgaaggatacgattctaaaatctataaagataccgaagggttttggactatcggtatcggtcaccttttaactcgcgacccttctcttgaagtagccaaaagagaacttgataaactcgttgggcgtaagtgcaacggtcaaatcactcagtcggaagcagaaaagattttcgctgatgatgttgataaagctattaatggaatcaagaaaaacgcttctctgaaaccggtttatgattctcttgatggagatgatccgcgtcaagctgcactcatcaatatggtatttcaaatgggtgtagctggtgtagctggcttcactaattctatgagaatggtaaaagaaaaacgttgggctgatgcagctgtaaatttagctcaatcaaaatggtatcgtcaaactccgaatcgcgctaaacgagtaattgaaacattccgcactggaacatggaacgcatataaa

SEQ ID NO: 9 - DNA, 29 bp, Artificial sequence
  ORF131 forward primer
gggcatatgaacatttttaaaatgcttcg

SEQ ID NO: 10 - DNA, 29 bp, Artificial sequence
  ORF131 reverse primer
gggggatcctcatttatatgcgttccatg

(19) United States
(12) Patent Application Publication
(10) Pub. No.: US 2019/0330609 A1
(43) Pub. Date: Oct. 31, 2019

(54) NOVEL ENDOLYSIN

(71) Applicant: UNIVERSIDADE DO MINHO, Braga (PT)

(72) Inventors: Hugo Alexandre MENDES DE OLIVEIRA, Braga (PT); Joana Joana VALENTE DE RODRIGUES AZEREDO, Braga (PT)

(21) Appl. No.: 16/469,800

(22) PCT Filed: Dec. 18, 2017
(86) PCT No.: PCT/EP2017/083326

§ 371 Date: Jun. 14, 2019

(30) Foreign Application Priority Data
Dec. 16, 2016 (EP) 16204770.8

(51) Int. Cl. C12N 9/36; A61P 31/04; C07K 19/00

(52) U.S. Cl. CPC C12N 9/2462; A61P 31/04; A23L 3/3571; C12Y 302/01017; C07K 2319/21; C07K 19/00

(57) ABSTRACT

The present invention relates to a polypeptide with endolysin activity and amino acid sequences according to SEQ ID No. 1 and fragments or derivatives thereof. Moreover, the present invention relates to nucleic acid molecules encoding said polypeptide, vectors comprising said nucleic acid molecules and host cells comprising either said nucleic acid molecules or said vectors. In addition, the present invention relates to said polypeptide for use as a medicament, in particular for the treatment or prevention of Gram-negative bacterial infections, as diagnostic means, as cosmetic substance or as sanitizing agent. The present invention also relates to the use of said polypeptide for the treatment or prevention of bacterial contamination, particularly of Gram-negative contamination, of foodstuff, of food processing equipment, of food processing plants, of surfaces coming into contact with foodstuff, of medical devices, of surfaces in hospitals and doctor's offices. Furthermore, the present invention relates to a pharmaceutical composition comprising said polypeptide.

### FIELD OF THE INVENTION

The present invention relates to a polypeptide with endolysin activity and amino acid sequences according to SEQ ID No. 1 and fragments or derivatives thereof. Moreover, the present invention relates to nucleic acid molecules encoding said polypeptide, vectors comprising said nucleic acid molecules and host cells comprising either said nucleic acid molecules or said vectors. In addition, the present invention relates to said polypeptide for use as a medicament, in particular for the treatment or prevention of Gram-negative bacterial infections, as diagnostic means, as cosmetic substance or as sanitizing agent. The present invention also relates to the use of said polypeptide for the treatment or prevention of bacterial contamination, particularly of Gram-negative contamination, of foodstuff, of food processing equipment, of food processing plants, of surfaces coming into contact with foodstuff, of medical devices, of surfaces in hospitals and doctor's offices. Furthermore, the present invention relates to a pharmaceutical composition comprising said polypeptide.

### BACKGROUND OF THE INVENTION

Life-threatening human infectious diseases caused by bacterial pathogens are now reemerging in part due to the increased resistance to antibiotics. Gram-negative bacteria correspond to a prominent part of drug resistant pathogens and have become a major public health threat leading to an increasing morbidity and mortality caused by infection diseases. Many efforts have been put forward but even so the rate of multidrug-resistant Gram-negative bacteria is still rising throughout the world and has become a growing problem. There is a scarcity or even inexistence of new active antibiotics specifically developed for multidrug-resistant Gram-negative bacteria in contrast with what happened with Gram-positive bacteria and consequently there are now a growing number of Gram-negative organisms isolated in clinical samples and environments classified as pan-drug resistant isolates, i.e., bacteria resistant to all available antibiotics.

The Gram-negative cell wall is constituted by an outer membrane which is a major obstacle to most antimicrobials. With a few exceptions, the outer membrane consists of an asymmetric membrane of phospholipids (inner leaflet), lipopolysaccharides (LPS) (outer leaflet) and proteins, like porins, serving as defusing channels, and lipoproteins, covalently bound to the peptidoglycan and anchored to the inner leaflet. The LPS is the major component of the outer membrane and provides the bacterium a permeability barrier for many external agents. The LPS is an extremely diverse structure that can be modified in response to prevailing environmental conditions (Nikaido, 2003). Three different regions can be distinguished according to their chemical structure, biosynthesis, genetics and function: i) the lipid A component, ii) the core region and iii) the O-antigen, also known as O-specific polysaccharides or O-side chains. The LPS may be presented in a rough (R-type, without O-antigen), smooth (S-type, with O-antigen) or a mixture (SR-type). The overall outer membrane integrity is maintained by two main stabilizing forces, the LPS cation-binding sites (ionic interactions) and the dense hydrophobic stacking of the Lipid A acyl chain groups (hydrophobic interactions), and act as an efficient diffusion barrier function as a natural barrier against antimicrobials. Thus, there is a need for new antimicrobial agents active against Gram-negative bacteria.

Endolysins used as ‘enzybiotics’—a hybrid term of ‘enzymes’ and ‘antibiotics’—perfectly met this need. Endolysins are specialized peptidoglycan-degrading enzymes encoded by all double stranded DNA bacteriophages (Young et al., Trends Microbiol. 2000 March; 8(3):120-128). Endolysin-mediated action is a coordinated and regulated event by other proteins to degrade and compromise the bacterial peptidoglycan (PG) layer at the end of the bacteriophage replication cycle. Depending on their activity and the bonds that they hydrolyse, endolysins can be divided into different classes: i) Glycosidases that cleave the glycan component at the reducing end of N-acetylglucosamine (GlcNAc) or at the reducing end of N-acetylmuramic acid (MurNAc); ii) N-acetyl-β-D-muramidases (often called lysozymes or muramidases) that share the same glycan target as the lytic transglycosidases, but deliver different end products; iii) amidases that catalyze the hydrolysis of the critical amide bond between MurNAc and the L-alanine, separating the glycan strand from the stem peptide; and iv) distinct classes of endopeptidases and carboxypeptidases that attack the LD- and DD-bonds in the stem peptides that crosslink the cell wall.

While the potential of endolysins has been known for a long time, their antibacterial use was overshadowed by the success and dominance of antibiotics. Only due to the alarming emergence of antibiotic resistant bacteria, scientific community and business players have revived their interest in endolysins as an alternative therapy. In 2001 the first endolysin study revealed a great capacity to destroy group A, C and E streptococci in seconds leaving 14 other commensal streptococci unharmed. This unique ability of endolysins to rapidly cleave PG in a species-specific manner when added exogenously, instigated researchers to isolate a huge number of endolysins to successfully target other Gram-positive pathogens. However, endolysins attacking Gram-negative cells, have received little attention mostly due to the presence of a highly protective outer membrane which the endolysins by itself cannot destroy in order to be able to act on the PG. As a result, an endolysin-based technology to target Gram-negative cells such as hospital and foodborne pathogens (e.g. Salmonella, Escherichia coli, Pseudomonas spp., Acinetobacter spp.) is perhaps one of the most important challenges in endolysin therapy today.

### SUMMARY OF THE INVENTION

The present invention relates to a novel polypeptide with peptidoglycan degrading activity, isolated from Citrobacter koseri phage CkP1, with antimicrobial activity against Gram-negative bacteria.

A first object of the present invention provides a polypeptide having endolysin activity comprising an amino acid sequence according to SEQ ID No. 1 or a fragment or derivative thereof. The polypeptide comprising an amino acid sequence according to SEQ ID No. 1 is preferably encoded by a nucleotide sequence according to SEQ ID No. 2. In a preferred embodiment said fragment comprises an amino acid sequence according to SEQ ID No. 3, which is preferably encoded by nucleotide sequence SEQ ID No. 4. In yet another preferred embodiment said derivative has a deletion, addition, insertion and/or substitution in the amino acid sequence according to SEQ ID No. 1 and SEQ ID No. 3. In yet another preferred embodiment, said polypeptide according to the present invention comprises at the N- or C-terminus a peptide stretch having membrane or LPS disrupting activity, in particular a cationic or polycationic peptide. Said polypeptide may additionally comprise a tag, preferably a His6-tag.

A second object of the present invention provides an isolated nucleic acid molecule encoding a polypeptide according to the invention.

A third object of the present invention relates to a vector comprising a nucleic acid molecule according to the present invention.

A fourth object of the present invention relates to a host cell comprising a nucleic acid molecule according to the present invention or a vector according to the present invention.

A fifth object of the present invention relates to the polypeptide according to the present invention for use in a method of treating the human or animal body by surgery or therapy and or for use in a diagnostic method practised on the human or animal body. In particular, the present invention relates to the polypeptide according to the present invention for use in a method of treating or preventing Gram-negative bacterial infections in a subject, such as a human or an animal. The polypeptide according to the present invention can also be used as diagnostic substance. For example, lysis of bacteria upon ex vivo contact with a polypeptide according to the present invention (e.g. in a bacterial culture established from a sample of a subject or from the environment) indicates presence of Gram-negative bacteria.

A sixth object of the present invention relates to the use of a polypeptide according to the present invention as an antimicrobial in food, feed or in cosmetics, or as a disinfecting agent. Another preferred embodiment relates to the use of said polypeptide according to the present invention for the treatment or prevention of Gram-negative bacterial contamination of foodstuff, of food processing equipment, of food processing plants, of (inanimate) surfaces coming into contact with foodstuff, of feed, of feed processing equipment, of feed processing plants, of (inanimate) surfaces coming into contact with feed, of medical devices, of (inanimate) surfaces in hospitals and doctor's offices.

### DETAILED DESCRIPTION

### Legends to the Figures

The following figures serve to illustrate the invention.

FIG. 1. Bioinformatics analysis of the wild type Citrobacter koseri endolysin CkP1gp131 using BLASTp and Pfam systems. The DNA and amino acid sequences as they appear in the wild type phage are A) SEQ ID No. 2 and B) SEQ ID No. 1, respectively. The catalytic domain of the lysozyme-like superfamily (amino acids 1-163, SEQ ID No. 3, underlined) is visualized in the scheme (FIG. 1 C) and the sequences (FIGS. 1 A and B; underline).

FIG. 2. Antibacterial activity of CkP1gp131 against several Gram-negative bacterial pathogens. Exponentially growing cells (≈3×10⁷ CFU/ml) were incubated for 2 hours and at 37° C., with 50 μg/ml (2.4 μM) of CkP1gp131 resuspended in 20 mM HEPES pH 7.0 or with HEPES as a negative control. After incubation, the antibacterial effect was assessed by quantification of the number of CFUs and expressed as relative inactivation in logarithmic units (=log 10 (N₀/Nᵢ) with N₀=number of untreated cells and Nᵢ=number of cells after treatment). Averages±standard deviations are given for n=3 repeats. CK—Citrobacter koseri strains (#1 to 11); CF—Citrobacter freundii strains (#1 to 7); * below the limit of detection (<10 cfu/ml).

FIG. 3. Antibacterial activity of CkP1gp131: A) Activity under different pH values. B) Activity under different ionic strength. Two concentrations of endolysin (5 and 50 μg/ml) were tested. Activity is expressed as relative inactivation in logarithmic units (=log 10 (N0/Ni) with N0=number of untreated cells and Ni=number of cells after treatment). Averages±standard deviations are given for n=4 repeats. * below the limit of detection (<10 CFU/ml).

FIG. 4. Antibacterial activity of CkP1gp131: A) Activity under different temperatures. Two concentrations of endolysin (5 and 50 μg/ml) were tested. Averages±standard deviations are given for n=4 repeats. B) Activity over a period of 30 days (50 μg/ml). Activity is expressed as relative inactivation in logarithmic units (=log 10 (N0/Ni) with N0=number of untreated cells and Ni=number of cells after treatment). * below the limit of detection (<10 CFU/ml).

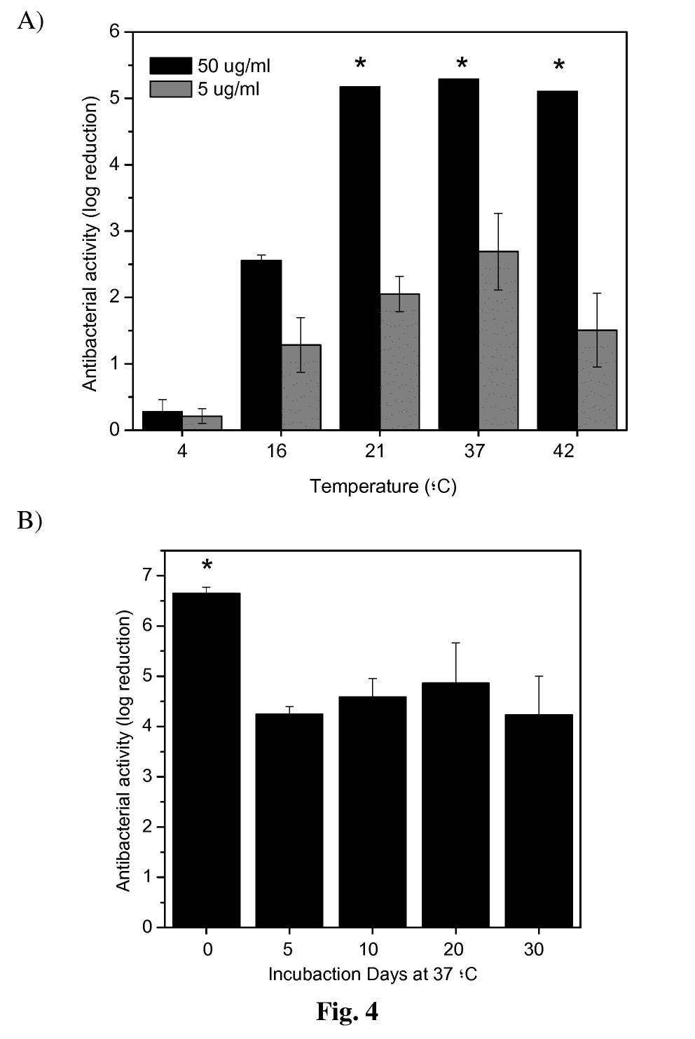

### DESCRIPTION

### Definitions

If appearing herein, the following terms shall have the definitions set out below.

The term “polypeptide” as used herein refers synonymously to the term “protein”. The term “polypeptide” as used herein refers to a linear polymer of amino acid residues linked by peptide bonds in a specific sequence. The amino acid residues of a protein may be modified by e.g. covalent attachments of various groups such as carbohydrates and phosphate. Other substances may be more loosely associated with the polypeptide chains, such as heme or lipid, giving rise to the conjugated proteins which are also comprised by the term “polypeptide” as used herein. The various ways in which the polypeptide chains fold have been elucidated, in particular with regard to the presence of alpha helices and beta-pleated sheets. The term “polypeptide” as used herein refers to all four classes of proteins being all-alpha, all-beta, alpha/beta and alpha plus beta.

The term “peptide stretch” as used herein refers to any kind of peptide linked to a protein such as an endolysin. However, a peptide stretch in the meaning of the present invention does not refer to His6-tags, Strep-tags, Avi-tags, Myc-tags, Gst-tags, JS-tags, cystein-tags, FLAG-tags or other tags known in the art, thioredoxin or maltose binding proteins (MBP). The term “tag” in contrast to the term “peptide stretch” as used herein refers to a peptide which can be useful to facilitate expression and/or affinity purification of a polypeptide, to immobilize a polypeptide to a surface or to serve as a marker or a label moiety for detection of a polypeptide e.g. by antibody binding in different ELISA assay formats as long as the function making the tag useful for one of the above listed facilitation is not caused by the positively charge of said peptide. However, the His6-tag may, depending on the respective pH, also be positively charged, but is used as affinity purification tool as it binds to immobilized divalent cations and is not used as a peptide stretch according to the invention.

The term “peptide stretch” as used herein refers to short polypeptide sequences consisting of from about 2 to 10 about 100 amino acid residues, more preferably from about 4 to about 50 amino acid residues, more preferably from about 5 to about 30 amino acid residues, wherein the amino group of one amino acid residue is linked to the carboxyl group of another amino acid residue by a peptide bond.

The term “endolysin activity” as used herein refers to the activity of hydrolysing bacterial cell walls, in particular by degrading bacterial peptidoglycan such as the peptidoglycan of Gram-negative bacteria.

The term “wild type” or “wt” as used herein refers to the amino acid sequence of the endolysin CkP1gp131 as depicted in SEQ ID No. 1. The nucleic acid sequence encoding the wild type endolysin CkP1gp131 is depicted in SEQ ID No. 2.

The term “deletion” as used herein refers preferably to the absence of 1, 2, 3, 4, 5 (or even more than 5) continuous amino acid or nucleic acid residues in the derivative sequence in comparison to the respective reference sequence. A derivative may exhibit one, two or more of such deletions.

The term “insertion” as used herein refers preferably to the additional intrasequential presence of 1, 2, 3, 4, 5 (or even more than 5) continuous amino acid or nucleic acid residues in the derivative sequence in comparison to the respective reference sequence. A derivative of the present invention may exhibit one, two or more of such insertions.

The term “addition” as used herein refers preferably to the additional presence of 1, 2, 3, 4, 5 (or even more than 5) continuous amino acid or nucleic acid residues at the N- and/or C-terminus of the derivative sequence in comparison to the respective reference sequence.

The term “substitution” as used herein refers to the presence of an amino acid or nucleic acid residue located at a certain position of the derivative sequence, which is different from the amino acid or nucleic acid residue which is present or absent at the corresponding position in the reference sequence. A derivative of the present invention may exhibit 1, 2, 3, 4, 5, 6, 7, 8, 9, 10, 11, 12, 13, 14, 15, 16, 17, 18, 19, 20, or more of such substitutions. As mentioned above, preferably such substitutions are conservative substitutions.

The term “cell wall” as used herein refers to all components that form the outer cell enclosure of the Gram-negative bacteria and thus guarantee their integrity. In particular, the term “cell wall” as used herein refers to peptidoglycan, the outer membrane of the Gram-negative bacteria with the lipopolysaccharide, the bacterial cell membrane, but also to additional layers deposited on the peptidoglycan as e.g. capsules, outer protein layers or slimes.

The present invention relates to a new endolysin, isolated from Citrobacter koseri phage CkP1, useful in the preparation of novel antibacterial agents against Gram-negative bacteria. In particular the present invention relates to a polypeptide comprising an amino acid sequence according to SEQ ID No. 1 or fragments or derivatives thereof. The polypeptide comprising an amino acid sequence according to SEQ ID No. 1 is preferably encoded by a nucleotide sequence according to SEQ ID No. 2.

The endolysin CkP1gp131 having an amino acid sequence according to SEQ ID No. 1 has a length of 164 amino acids. The enzymatic active domain (EAD) (aa 1 to 163) complies with the lysozyme-like superfamily domain motif with an amino acid sequence according to SEQ ID No. 3 and a nucleotide sequence according to SEQ ID No. 4.

Thus, preferred fragments of SEQ ID No. 1 are polypeptides comprising an amino acid sequence according to SEQ ID No. 3. Other preferred fragments of SEQ ID No. 1 are polypeptides lacking the N-terminal methionine of CkP1gp131 (e.g. see SEQ ID No. 5 and SEQ ID No. 6). Preferably, fragments of the polypeptide according to the present invention are at least 80, more preferably at least 100, even more preferably at least 120, even more preferably at least 140, most preferably at least 160 amino acids long. Preferably, a polypeptide according to the present invention comprising a fragment of SEQ ID No. 1 does not comprise the sequence of SEQ ID No. 1. Preferably, a polypeptide according to the present invention comprising a fragment of SEQ ID No. 1 exhibits essentially the lytic activity of the wt CkP1gp131 endolysin (SEQ ID No. 1). Said activity represents preferably about 10, 20, 30, 40, 50, 60, 70, 80, 90, 100, 110, 120, 130, 140, 150, 160, 170, 180, 190 or about 200% of the activity of the wt CkP1gp131 endolysin.

In some embodiments, the polypeptide of the present invention comprises an amino acid sequence, which is a derivative of the amino acid according to SEQ ID No. 1. Such derivatives may be polypeptides comprising essentially an amino acid sequence according to SEQ ID No. 1, SEQ ID No. 3, SEQ ID No. 5 and/or SEQ ID No. 6 but having additional modifications and/or alterations.

In one embodiment, said modifications and/or alterations of said derivatives of the endolysin according to the present invention can be mutations, in particular deletions, insertions, additions, substitutions or any combinations thereof. Said derivatives according to the present invention exhibit essentially the lytic activity of the wt CkP1gp131 endolysin (SEQ ID No. 1). Said activity represents preferably about 10, 20, 30, 40, 50, 60, 70, 80, 90, 100, 110, 120, 130, 140, 150, 160, 170, 180, 190 or about 200% of the activity of the wt CkP1gp131 endolysin.

The activity of polypeptides comprising fragments and derivatives of wt CkP1gp131 endolysin (SEQ ID No. 1) can be measured by assays well known in the art by a person skilled in the art as e.g. the plate lysis assay or the liquid lysis assay which are e.g. described in (Briers et al., J. Biochem. Biophys 20 Methods 70: 531-533, (2007)).

In a preferred embodiment of the present invention the polypeptide according to the present invention comprises additionally a tag such as a His6-tag, Strep-tag, Avi-tag, Myc-tag, Gst-tag, JS-tag, cystein-tag, FLAG-tag or other tags known in the art at the N-terminus or at the C-terminus. In a preferred embodiment of the present invention said tag is linked to the sequence of SEQ ID No. 1, or the fragment and/or derivative thereof, at the C-terminus (e.g. N-tag-SEQ ID No. 1-C). Said tag may be linked to SEQ ID No. 1, or the fragment or derivative thereof over additional amino acid residues. Said additional amino acid residues may consist of at least 1, 2, 3, 4, 5, 6, 7, 8, 9 or 10 additional amino acid residues. In a preferred embodiment of the present invention the tag is linked to SEQ ID No. 1, the fragment or the derivative by the additional amino acid residues Leu-Glu or Lys-Gly. In a preferred embodiment the present invention relates to a polypeptide comprising an amino acid sequence according to SEQ ID No. 7. The polypeptide having an amino acid sequence according to SEQ ID No. 7 comprises in comparison to the polypeptide having an amino acid sequence according to SEQ ID No. 1, respectively, an additional His6-tag linked to the N-terminus of SEQ ID No.1. A polypeptide comprising an amino acid sequence according to SEQ ID No. 7 is preferably encoded by a nucleotide sequence according to SEQ ID No. 8.

The present invention further relates to an isolated nucleic acid molecule encoding a polypeptide according to the present invention. Especially preferred isolated nucleic acid molecules according to the present invention comprise a nucleic acid sequence according to SEQ ID No. 2, SEQ ID No. 4 and/or SEQ ID No. 8. Preferably, the nucleic acid molecule comprises a promotor heterologous to SEQ ID No. 1.

The present invention further relates to a vector comprising the nucleic acid molecule according to the present invention. Said vector may provide for the constitutive or inducible expression of said polypeptide according to the present invention.

The invention also relates to a host cell comprising a polypeptide, nucleic acid and/or vector according to the present invention. The host cell may be a genetically modified suitable host cell which expresses said polypeptide. Said host cell may be a micro-organism such as bacteria or yeast or an animal cell as e.g. a mammalian cell, in particular a human cell. A host cell according to the present invention is not part of a multicellular organism but isolated (e.g., an immortalized cell line etc.). In one embodiment of the present invention the host cell is an Escherichia coli cell. The host may be selected due to mere biotechnological reasons, e.g. yield, solubility, costs, etc. but maybe also selected from a medical point of view, e.g. a nonpathological bacteria or yeast or human cells.

Another aspect of the present invention is related to a method for genetically transforming a suitable host cell in order to obtain the expression of the polypeptide according to the present invention, wherein the host cell is genetically modified by the introduction of a nucleic acid according to the present invention encoding said polypeptide according to the present invention into the host cell. Translation and expression of the polypeptide according to the present invention can be obtained by genetic engineering methods well known by the man skilled in the art.

The invention also relates to a method for obtaining a polypeptide according to the present invention from a host cell according to the present invention, such as a genetically modified suitable host cell which expresses said polypeptide, wherein the method comprises isolating the a polypeptide according to the present invention from said host cell.

In a further aspect the present invention relates to a composition, preferably a pharmaceutical composition, comprising a polypeptide according to the present invention and/or a host cell transformed with a nucleic acid molecule or a vector according to the present invention, and further comprising a diluent, excipient or carrier, such as a pharmaceutical acceptable diluent, excipient or carrier.

In a preferred embodiment of the present invention the composition comprises additionally agents permeabilizing the outer membrane of Gram-negative bacteria such metal chelators as e.g. EDTA, TRIS, lactic acid, lactoferrin, polymyxin, citric acid and/or other substances as described e.g. by Vaara (Agents that increase the permeability of the outer membrane. Vaara M. Microbiol Rev. 1992 September; 56(3):395-441). Also preferred are compositions comprising combinations of the above mentioned permeabilizing agents. Especially preferred is a composition comprising about 10 μM to about 100 mM EDTA, more preferably about 50 μM to about 10 mM EDTA, more preferably about 0.5 mM to about 10 mM EDTA, more 30 preferably about 0.5 mM to about 2 mM EDTA, more preferably about 0.5 mM to about 1 mM EDTA. Also preferred is a composition comprising about 0.5 mM to about 2 mM EDTA, more preferably about 1 mM EDTA and additionally about 10 to about 100 mM TRIS.

The present invention also relates to a polypeptide according to the present invention and/or a host transformed with a nucleic acid comprising a nucleotide sequence encoding a polypeptide according to the present invention for use in a method of treating the human or animal body by surgery or therapy and diagnostic methods practised on the human or animal body. In a further aspect the present invention relates to the use of a polypeptide according to the present invention and/or a host transformed with a vector comprising a nucleic acid molecule comprising a nucleotide sequence encoding a polypeptide according to the present invention in a method for the treatment and/or prevention of a disorder, disease or condition associated with Gram-negative bacteria. In particular the disorder, disease or condition may be caused by Gram-negative bacteria of bacterial groups, families, genera or species comprising strains pathogenic for humans or animals like Enterobacteriaceae (Escherichia, especially E. coli, Salmonella, Shigella, Citrobacter, Edwardsiella, Enterobacter, Hafnia, Klebsiella, especially K. pneumoniae, Morganella, Proteus, Providencia, Serratia, Yersinia), Pseudomonadaceae (Pseudomonas, especially P. aeruginosa), Burkholderia, Stenotrophomonas, Shewanella, Sphingomonas, Comamonas, Neisseria, Moraxella, Vibrio, Aeromonas, Brucella, Francisella, Bordetella, Legionella, Bartonella, Coxiella, Haemophilus, Pasteurella, Mannheimia, Actinobacillus, Gardnerella, Spirochaetaceae (Treponema and Borrelia), Leptospiraceae, Campylobacter, Helicobacter, Spirillum, Streptobacillus, Bacteroidaceae (Bacteroides, Fusobacterium, Prevotella, Porphyromonas), Acinetobacter, especially A. baumannii. In particular, the treatment and/or prevention of the disorder, disease or condition may be caused by Pseudomonas aeruginosa, Pseudomonas putida, Burkholderia pseudomallei, E. coli and/or Salmonella Typhimurium.

In a further aspect the present invention relates to a method of treating a disorder, disease or condition in a subject in need of treatment and/or prevention, which method comprises administering to said subject an effective amount of a polypeptide according to the present invention and/or an effective amount of a host transformed with a nucleic acid comprising a nucleotide sequence encoding a polypeptide according to the present invention or an effective amount of a composition according to the present invention. The subject may for example be a human or an animal. In particular said method of treatment may be for the treatment and/or prevention of infections of the skin, of soft tissues, the respiratory system, the lung, the digestive tract, the eye, the ear, the teeth, the nasopharynx, the mouth, the bones, the vagina, of wounds of bacteraemia and/or endocarditis caused by Gram-negative bacteria, in particular by the Gram-negative bacteria as listed above.

The dosage and route of administration used in a method of treatment (or prophylaxis) according to the present invention depends on the specific disease/site of infection to be treated. The route of administration may be for example oral, topical, nasopharyngeal, parenteral, intravenous, rectal or any other route of administration. For application of a polypeptide according to the present invention and/or an effective amount of a host transformed with a nucleic acid comprising a nucleotide sequence encoding a polypeptide according to the present invention or a composition according to the present invention to a site of infection (or site endangered to be infected) a formulation may be used that protects the active compounds from environmental influences such as proteases, oxidation, immune response etc., until it reaches the site of infection. Therefore, the formulation may be capsule, dragee, pill, suppository, injectable solution or any other medical reasonable galenic formulation. Preferably, the galenic formulation may comprise suitable carriers, stabilizers, flavourings, buffers or other suitable reagents. For example, for topical application the formulation may be a lotion or plaster, for nasopharyngeal application the formulation may be saline solution to be applied via a spray to the nose.

Preferably, a polypeptide according to the present invention is used for medical treatment, if the infection to be treated (or prevented) is caused by multiresistant bacterial strains, in particular by strains resistant against one or more of the following antibiotics: streptomycin, tetracycline, cephalothin, gentamicin, cefotaxime, cephalosporin, ceftazidime or imipenem. Furthermore, a polypeptide according to the present invention can be used in methods of treatment by administering it in combination with conventional antibacterial agents, such as antibiotics, lantibiotics, bacteriocins or endolysins, etc.

The present invention also relates to a pharmaceutical pack comprising one or more compartments, wherein at least one compartment comprises one or more polypeptides according to the present invention, one or more hosts transformed with a nucleic acid comprising a nucleotide sequence encoding a polypeptide according to the present invention and/or a composition according to the present invention.

In another aspect the present invention relates to a process of preparation of a pharmaceutical composition, said process comprising admixing one or more polypeptides according to the present invention and/or one or more hosts transformed with a nucleic acid comprising a nucleotide sequence encoding a polypeptide according to the present invention with a pharmaceutically acceptable diluent, excipient or carrier.

In an even further aspect the composition according to the present invention is a cosmetic composition. Several bacterial species can cause irritations on environmentally exposed surfaces of the patient's body such as the skin. In order to prevent such irritations or in order to eliminate minor manifestations of said bacterial pathogens, special cosmetic preparations may be employed, which comprise sufficient amounts of the polypeptide according to the present invention in order to degrade already existing or freshly settling pathogenic Gram-negative bacteria.

In a further aspect the present invention relates to a polypeptide according to the present invention for use as diagnostic means in medicinal diagnostics, food diagnostics, feed diagnostics or environmental diagnostics, in particular as a diagnostic means for the diagnostic of bacterial infection or contamination caused in particular by Gram-negative bacteria. In this respect the polypeptide according to the present invention may be used as a tool to specifically degrade pathogenic bacteria, in particular Gram-negative pathogenic bacteria. The degradation of the bacterial cells by the polypeptide according to the present invention can be supported by the addition of detergents like Triton X-100 or other additives which weaken the bacterial cell envelope like polymyxin B. Specific cell degradation is needed as an initial step for subsequent specific detection of bacteria using nucleic acid based methods like PCR, nucleic acid hybridization or NASBA (Nucleic Acid Sequence Based Amplification), immunological methods like IMS, immunofluorescence or ELISA techniques, or other methods relying on the cellular content of the bacterial cells like enzymatic assays using proteins specific for distinct bacterial groups or species (e.g. β-galactosidase for enterobacteria, coagulase for coagulase positive strains).

In a further aspect the present invention relates to the use of the polypeptide according to the present invention for the treatment or prevention of Gram-negative bacterial contamination of foodstuff, of food processing equipment, of food processing plants, of (inanimate) surfaces coming into contact with foodstuff such as shelves and food deposit areas and in all other situations, where pathogenic, facultative pathogenic or other undesirable bacteria can potentially infest food material. Likewise, the polypeptide according to the present invention may be used for the treatment or prevention of Gram-negative bacterial contamination of feedstuff, of feed processing equipment, of feed processing plants, of (inanimate) surfaces coming into contact with feedstuff such as shelves and feed deposit areas and in all other situations, where pathogenic, facultative pathogenic or other undesirable bacteria can potentially infest feed material. The polypeptide according to the present invention may also be used for the treatment or prevention of Gram-negative bacterial contamination of medical devices and of all kinds of (inanimate) surfaces in hospitals and doctor's offices.

In particular, a polypeptide of the present invention may be used prophylactically as sanitizing agent. Said sanitizing agent may be used before or after surgery, or for example during hemodialysis. Moreover, premature infants and immunocompromised persons, or those subjects with need for prosthetic devices may be treated with a polypeptide according to the present invention. Said treatment may be either prophylactically or during acute infection. In the same context, nosocomial infections, especially by antibiotic resistant strains like Pseudomonas aeruginosa (FQRP), Acinetobacter species and Enterobacteriaceae such as E. coli, Salmonella, Shigella, Citrobacter, Edwardsiella, Enterobacter, Hafnia, Klebsiella, Morganella, Proteus, Providencia, Serratia and Yersinia species may be treated prophylactically or during acute phase with a polypeptide of the present invention. Therefore, a polypeptide according to the present invention may be used as a disinfectant also in combination with other ingredients useful in a disinfecting solution like detergents, tensids, solvents, antibiotics, lantibiotics, or bacteriocins.

The following examples explain the present invention but are not considered to be limiting to the disclosure or the appended claims. Unless indicated differently, molecular biological standard methods were used, as e.g., described by Sambrock et al., 1989, Molecular Cloning: A Laboratory Manual, 2nd edition, Cold Spring Harbor Laboratory Press, Cold 15 Spring Harbor, N.Y.

### Example 1. Cloning, Expression and Purification of the Endolysin CkP1gp131 of the Citrobacter koseri Phage CkP1

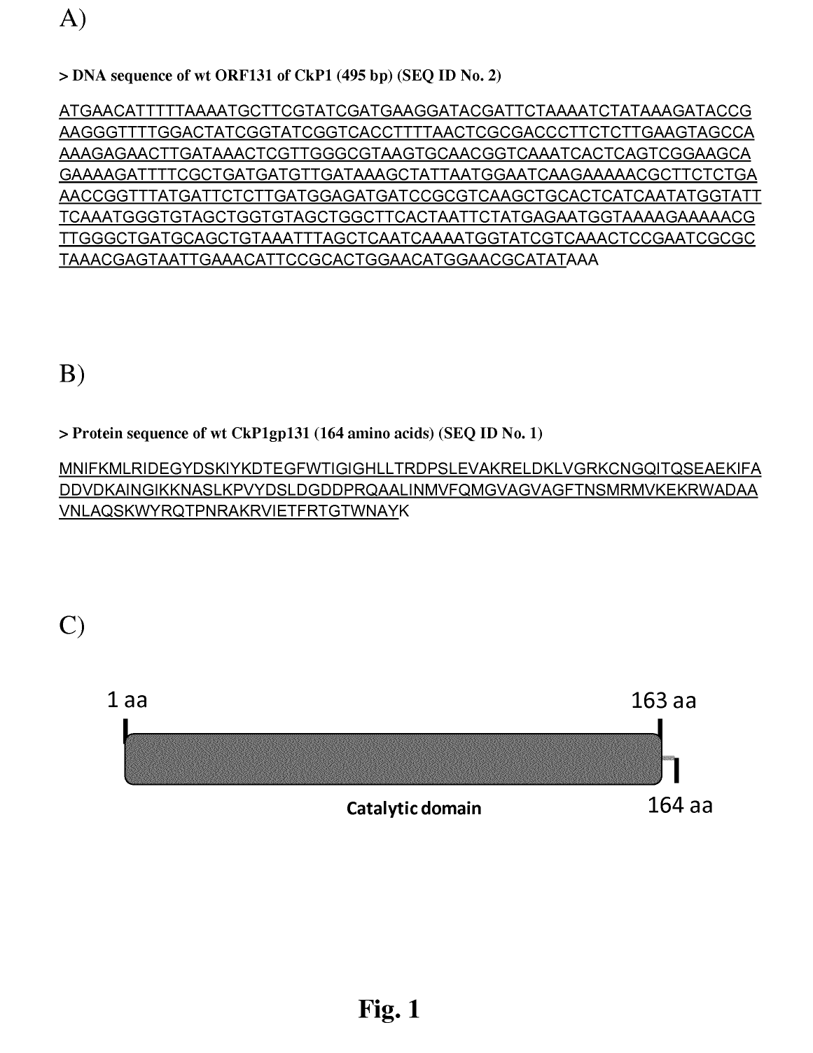

CkP1gp131 (164 amino acids long, MW=18609 Da; SEQ ID No. 1) is a globular endolysin originating from Citrobacter koseri phage CkP1 predicted to possess a lysozyme-like superfamily catalytic domain (from amino acids 1 to 163; SEQ ID No. 3) (see FIG. 1).

Purified genomic DNA of phage CkP1 was used as a template for the amplification of the open reading frame ORF131 encoding the endolysin CkP1gp131 in a standard PCR reaction with KAPA HiFi polymerase (Kapa Biosystems) using the following PCR parameters:

Start: 95° C., 2 min; followed by 35 cycles of [98° C. (20 sec)→60° C. (15 sec)→72° C. (20 sec)], followed by 72° C. (5 min) & cooling to 4° C. & storage.

Primers used during standard PCR amplification of ORF131 encoding endolysin CkP1gp131 were:

ORF131 forward primer
(SEQ ID No. 9)
5′ gggCATATGAACATTTTTAAAATGCTTCG 3′
ORF131 reverse primer
(SEQ ID No. 10)
5′ gggGGATCCTCATTTATATGCGTTCCATG 3′

The obtained PCR fragment was then ligated in the commercial available pET28a expression vector (Novagen). The obtained amino acid & DNA sequences for the recombinant CkP1gp131 are listed in SEQ ID No. 7 and SEQ ID No. 8, respectively.

Recombinant expression of CkP1gp131 was performed in exponentially growing E. coli BL21 (λDE3) cells after induction with 0.5 mM IPTG (isopropylthiogalactoside) at 37° C. for a period of 4 hours. The endolysin was then applied to Ni²⁺-NTA resin stacked in HisTrap™ HP 1 ml columns (GE Healthcare) for purification, using the N-terminal 6×His-tag, encoded by the pET28a expression vector. The purification process envolved four steps:

1. Equilibrium step—10 mL of washing buffer (25 mM imidazole, 0.5 mM NaCl and 20 mM NaH₂PO₄— NaOH on pH 7.4 concentration).

2. Loading step—Loading of total soluble expressed E. coli extract that was resuspended in washing buffer (25 mM imidazole, 0.5 mM NaCl and 20 mM NaH₂PO₄— NaOH on pH 7.4 concentration).

3. Washing step—10 mL of washing buffer (25 mM imidazole, 0.5 mM NaCl and 20 mM NaH₂PO₄— NaOH on pH 7.4 concentration).

4. Elution step—2 mL of elution buffer (250 mM imidazole, 0.5 mM NaCl and 20 mM NaH₂P0₄—NaOH on pH 7.4 concentration).

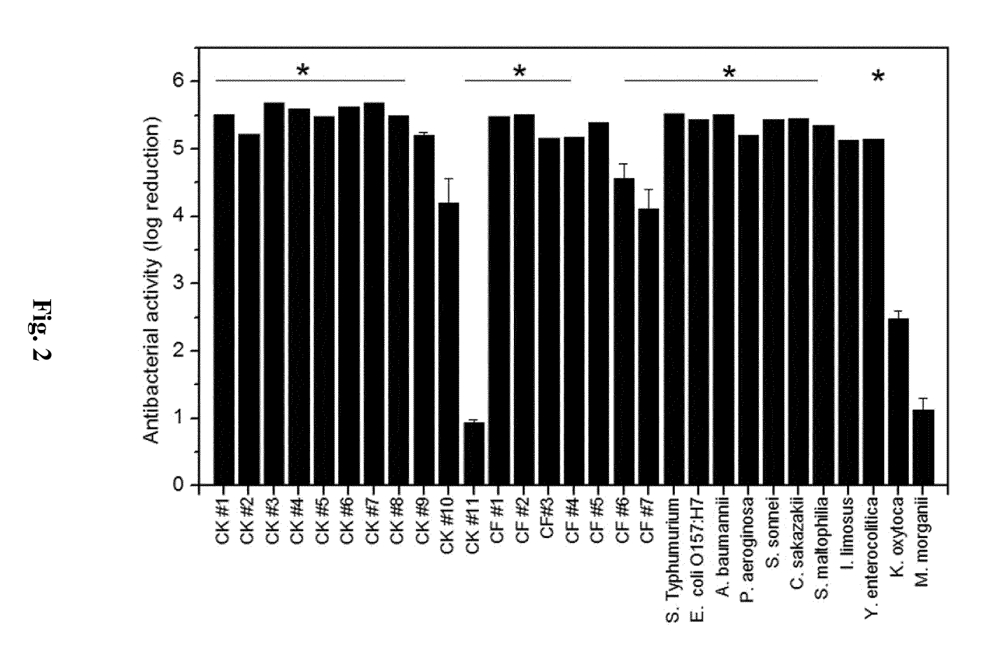

Eluted protein fractions of CkP1gp131 (MW=20772 Da) were visualized by standard denaturation SDS-PAGE gel (FIG. 2), achieving purities superior than 95%. Proteins were dialyzed in 10 mM HEPES at pH 7.0 (using Maxi GeBAflex-tube Dialysis Kit—Gene Bio-Application L.T.D) and concentration determined by the BCA™ Protein Assay Kit with bovine serum albumin as standard (Thermo Scientific). An expression yield of 37.2 mg per liter E. coli expression culture was obtained.

### Example 2. Antibacterial Effect of Citrobacter koseri Phage CKP1 Endolysin (SEQ ID No. 7) Against a Panel of Gram-Negative Pathogenic Bacteria

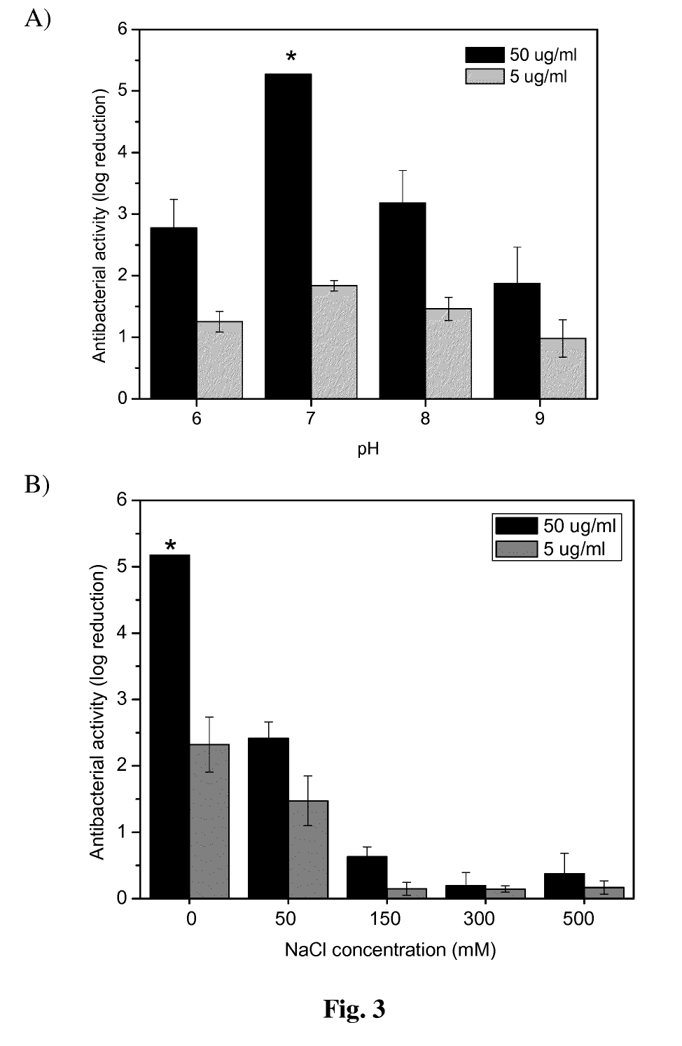

For the evaluation of the antibacterial effect, all antibacterial experiments were performed using 50 μg/ml of recombinant CkP1gp131 (SEQ ID No. 7) against mid-exponential phase cells in 10 mM HEPES (pH of 7.0) and incubated for 37° C. for 2 hours (FIG. 3).

The relative inactivation in logarithmic units (=log₁₀ (N₀/Nᵢ) with N₀=number of untreated cells (in the negative control) and Nᵢ=number of treated cells counted after incubation) is used to quantify the antibacterial activity. Averages±standard deviations are given for n=4 repeats.

Using the upper formula, CkP1gp131 possesses an antibacterial activity against all tested Gram-negative bacteria, ranging from 1 up to >5 logs (reaching the limit of detection of <100 cfu/ml).

### Example 3. Antibacterial Effect of Citrobacter koseri Phage CKP1 Endolysin (SEQ ID No. 7) Against Citrobacter koseri Under Various pH Conditions

Exponentially growing C. koseri cells (≈3×10⁹ CFU/ml) were 100× diluted into 20 mM of different buffer systems: Sodium citrate (pH 6); HEPES (pH 7-8) and Boric acid (pH 9). Reaction started by mixing 50 μL of cells with 50 μL of recombinant endolysin (or Hepes buffer as negative control) in order to have 5 or 50 μg/ml end concentration that. After incubation for 2 hours, the antibacterial effect was assessed by quantification of the number of CFUs and expressed as relative inactivation in logarithmic units (=log 10 (N₀/Nᵢ) with N₀=number of untreated cells and Nᵢ=number of cells after treatment).

### Example 4. Antibacterial Effect of Citrobacter koseri Phage CKP1 Endolysin (SEQ ID No. 7) Against Citrobacter koseri Under Various Ionic Strength Conditions

Exponentially growing C. koseri cells (≈3×10⁹ CFU/ml) were 100× diluted into 20 mM of HEPES pH 7 and were incubated with 5 or 50 μg/ml of endolysin (end concentration) for 2 hours, with increasing amounts of NaCl (0-500 mM). After incubation for 2 hours, the antibacterial effect was assessed by quantification of the number of CFUs and expressed as relative inactivation in logarithmic units (=log 10 (N₀/Nᵢ) with N₀=number of untreated cells and Nᵢ=number of cells after treatment).

### Example 5. Antibacterial Effect of Citrobacter koseri Phage CKP1 Endolysin (SEQ ID No. 7) Against Citrobacter koseri Under Various Temperature Conditions

Exponentially growing C. koseri cells were incubated with the endolysin using 20 mM of HEPES buffer, pH 7.0; 0 mM NaCl. After incubation for 2 hours, the antibacterial effect was assessed by quantification of the number of CFUs and expressed as relative inactivation in logarithmic units (=log 10 (N₀/Nᵢ) with N₀=number of untreated cells and Nᵢ=number of cells after treatment).

### Example 6. Antibacterial Effect of Citrobacter koseri Phage CKP1 Endolysin (SEQ ID No. 7) Against Citrobacter koseri Over Time

The endolysin (SEQ ID No. 7) was kept at 37° C. for several days in 20 mM Hepes pH 7.0 buffer. a) At day 0, 5 10, 20 and 30, 50 μg/ml of endolysin was a) tested against exponentially growing C. koseri cells (≈3×10⁷ CFU/ml) at 37° C. and for 2 hours, using again HEPES buffer as a negative control. After incubation, the antibacterial effect was assessed by quantification of the number of CFUs and expressed as relative inactivation in logarithmic units (=log 10 (N₀/Nᵢ) with N₀=number of untreated cells and Nᵢ=number of cells after treatment).

At day 0, 5 10, 20 and 30, the same sample was analysed on circular dichroism spectra in the far- and near-UV region (190 to 260 nm). The endolysin demonstrated to be highly stable, as no significant secondary structure changes were observed. Therefore, the CD thermal stability analysis demonstrates that the endolysin activity is still present after one month at 37° C.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A polypeptide comprising an amino acid sequence according to SEQ ID No. 1 or a fragment or derivative thereof.

2. The polypeptide according to claim 1, wherein the fragment comprises an amino acid sequence according to SEQ ID No. 3.

3. The polypeptide according to claim 1, wherein the derivative has a deletion, addition, insertion and/or substitution in the amino acid sequence according to SEQ ID No. 1, 3, 5, 6 and/or 7.

4. The polypeptide according to claim 1, wherein the polypeptide is fused at the N- or C-terminus to a peptide stretch having membrane or LPS disrupting activity.

5. The polypeptide according to claim 1, further comprising a tag, preferably a His6-tag.

6. The polypeptide according to claim 1, wherein the polypeptide has endolysin activity.

7. An isolated nucleic acid molecule encoding a polypeptide according to claim 1.

8. A vector comprising the nucleic acid molecule according to claim 7.

9. A host cell comprising the nucleic acid molecule according to claim 7.

10. A method of diagnosis practised on the human or animal body comprising administering to said human or animal body the polypeptide according to claim 1.

11. A food product, foodstuff, a cosmetic product or a disinfecting agent comprising the polypeptide accorind to claim 1.

12. A method for treating or preventing Gram-negative bacterial infection comprising contacting a subject having or at risk of Gram-negative bacterial infection with the polypeptide according to claim 1.

13. A method of treating or preventing Gram-negative bacterial contamination of foodstuff, of food processing equipment, of food processing plants, of surfaces coming into contact with foodstuff, of feed, of feed processing equipment, of feed processing plants, of surfaces coming into contact with feed, of medical devices, of surfaces in hospitals and doctor's offices comprising contact said foodstuff, of food processing equipment, of food processing plants, of surfaces coming into contact with foodstuff, of feed, of feed processing equipment, of feed processing plants, of surfaces coming into contact with feed, of medical devices, of surfaces in hospitals and doctor's offices with the polypeptide according to claim 1.

14. A pharmaceutical composition comprising the polypeptide according to claim 1 and a pharmaceutically acceptable diluent or excipient.

15. A host cell comprising the vector according to claim 8.
